(19) United States
(12) Patent Application Publication
(10) Pub. No.: US 2008/0182920 A1
(43) Pub. Date: Jul. 31, 2008

(54) Bone cement

(76) Inventors: Mark Robert Towler, County Clare (IE); Daniel Boyd, County Cork (IE); Owen Clarkin, County Wexford (IE)

Correspondence Address: JACOBSON HOLMAN PLLC, 400 SEVENTH STREET N.W., SUITE 600, WASHINGTON, DC, 20004, US

(21) Appl. No.: 12/010,480

(22) Filed: Jan. 25, 2008

Related U.S. Application Data
(60) Provisional application No. 60/897,464, filed on Jan. 26, 2007.

(51) Int. Cl. A61K 6/06; A61L 24/00

(52) U.S. Cl. 523/116; 106/35; 106/814; 106/810

(57) ABSTRACT

A bone cement comprises a zinc-based glass and polyalkenoate acid. In addition, the cement comprises a controlled amount of tri-sodium citrate (Na₃C₆H₅O₇) (“TSC”). The cement has enhanced rheology without negatively impacting on the mechanical properties. Controlled addition of TSC significantly improves the rheology of the bone cements, increasing working and setting times (illustrated in FIG. 1). For three formulations of the working time can be adjusted from less than 50 seconds to almost 120 seconds; concomitantly working times improve from 58 seconds to duration in excess of 300 s.

### FIELD OF THE INVENTION

The invention relates to bone cements for bone or dental implants.

### PRIOR ART DISCUSSION

The suitability of conventional aluminium containing GPCs (glass polyalkenoate cements) for skeletal applications is retarded by the presence, in the glass phase, of the aluminium ion (Al³⁺), a neurotoxin.

Also, GB2264711 describes a GPC having a zinc-containing silicate glass, and our patent specification no. WO2007/020613 describes an Al-free glass and cement.

The invention is directed towards providing an improved cement, particularly with one or more improved rheology property.

### REFERENCES

1. ‘The Processing, Mechanical Properties and Bioactivity of Zinc Based Glass Ionomer Cements’; D. Boyd & M. R. Towler; J. Mat. Sci: Materials in Medicine 16/9 (2005) p843-850.
2. ‘Zinc-based Glass Polyalkenoate Cements with Improved Setting Times and Mechanical Properties.’; D. Boyd, A. Wren, O. M. Clarkin & M. R. Towler; (accepted by) Acta Biomaterialia. (2007).

### SUMMARY OF THE INVENTION

According to the invention, there is provided a bone or dental cement comprising a glass and an acid, wherein the cement comprises a biocompatible agent capable of chelating ions released from the glass network during a setting process.

In one embodiment, the agent comprises a surfactant.

In another embodiment, the agent comprises a water soluble citrate salt. In a further embodiment, the agent comprises trisodium citrate, Na₃C₆H₅O₇.

In one embodiment, the agent comprises calcium citrate.

In another embodiment, the agent is present in a proportion of up to 30 wt %. In a further embodiment, the agent is present in a proportion of 5 wt % to 10 wt %.

In one embodiment, the glass comprises zinc as either a network former or a network modifier.

In another embodiment, the glass comprises ZnO.

In a further embodiment, the glass comprises SrO.

In one embodiment, the glass comprises CaO.

In another embodiment, the glass comprises SiO₂.

In a further embodiment, the acid comprises a water soluble polyalkenoic acid.

In one embodiment, the acid is poly-acrylic acid.

In another embodiment, the acid is present at a concentration in the range of 20% m/w to 60% m/w.

In a further embodiment, the agent is separate from the glass.

In one embodiment, the agent is incorporated in the glass.

In another aspect, there is provided a method of producing a bone cement, the cement having a target setting time, the method comprising the steps of producing a cement as defined above in which the agent concentration is chosen according to the target setting time.

In another aspect, there is provided a method of producing a bone cement, the cement having a target working time, the method comprising the steps of producing a cement as defined above in which the agent concentration is chosen according to the target working time.

In one embodiment, the agent is added as a powder to the glass and the acid before mixing with water.

### DETAILED DESCRIPTION OF THE INVENTION

### BRIEF DESCRIPTION OF THE DRAWINGS

The invention will be more clearly understood from the following description of some embodiments thereof, given by way of example only with reference to the accompanying drawings in which:—

FIG. 1 is a plot of working and setting times for a cement of the invention;

FIG. 2 is a set of plots of biaxial flexural strength v. maturation time; and

FIG. 3 is a set of plots of compressive strength v. maturation time.

### DESCRIPTION OF THE EMBODIMENTS

A bone cement comprises a zinc-based glass and polyalkenoate acid. In addition, the cement comprises a controlled amount of tri-sodium citrate (Na₃C₆H₅O₇) (“TSC”). The cement has enhanced rheology without negatively impacting on the mechanical properties.

### Cement Preparation

### Glass Synthesis

A series of glasses were produced (Table 1). Appropriate amounts of analytical grade silica, zinc oxide, calcium carbonate, and strontium carbonate (Sigma Aldrich, Dublin, Ireland), were weighed out in a plastics tub and mixed in a ball mill for one hour, then dried in a vacuum oven (100° C., 1 hr). The glass batches were then transferred to mullite crucibles for firing (1480° C., 1 hr). The glass melts were shock quenched into water and the resulting frits were dried, ground, and sieved to retrieve a <45 μm glass powder, which was used to form the cements.

TABLE 1
Glass Compositions (mol %)

| | SrO | CaO | ZnO | SiO₂ |
|---|---|---|---|---|
| BT100 | 0 | 0.16 | 0.36 | 0.48 |
| BT101 | 0.04 | 0.12 | 0.36 | 0.48 |
| BT102 | 0.08 | 0.08 | 0.36 | 0.48 |

### Poly(Acrylic) Acid

Ciba specialty polymers (Bradford, UK) supplied PAA (Mw, 80,800) in aqueous solution (25% m/w) was used. The PAA was freeze-dried, ground, and sieved to retrieve a <90 μm powder.

### Cement Preparation

Cements were prepared by thoroughly mixing the respective glass powders (<45 μm) with the appropriate amounts of PAA and distilled water on a glass plate (cement formulations illustrated in Table 2). Complete mixing was undertaken within 30 seconds. The concentrations of the PAA solutions are expressed in percent by mass (grams of solute/total grams of solution).

TABLE 2
Cement formulations examined in this study.

| Cement | Glass | PAA | Water |
|---|---|---|---|
| 40 wt % | 1 g | 0.3 g | 0.45 ml |
| 45 wt % | 1 g | 0.33 g | 0.41 ml |
| 50 wt % | 1 g | 0.37 g | 0.37 ml |

A second series of modified GPCs (mGPCs) was produced (Table 3) to examine the effect of TSC (Reagecon, Ireland) on the rheology and mechanical properties of the BT100 50 wt % (third row of Table 2) cement formulation. The modified GPC series was produced by addition of 5, 10 and 15 wt % additions of TSC to the base formulation of BT100 50 wt % GPC.

TABLE 3
TSC quantities used to create sub-series of
BT100 50 wt % GPC formulations.

| TSC | Equivalent addition |
|---|---|
| (g) | (wt %) |
| 0 | 0 |
| 0.0375 | 5 |
| 0.075 | 10 |
| 0.11 | 15 |

The mGPC series was produced by addition of 5, 10 and 15 wt % powdered TSC to the base formulation of BT100 50 wt % GPC. The TSC was added as a powder to the glass and acid before mixing with the water. TSC is a powder and can be mixed in with the other powdered reagents i.e. the acid and the glass, prior to be mixed with water.

### Improvements Achieved

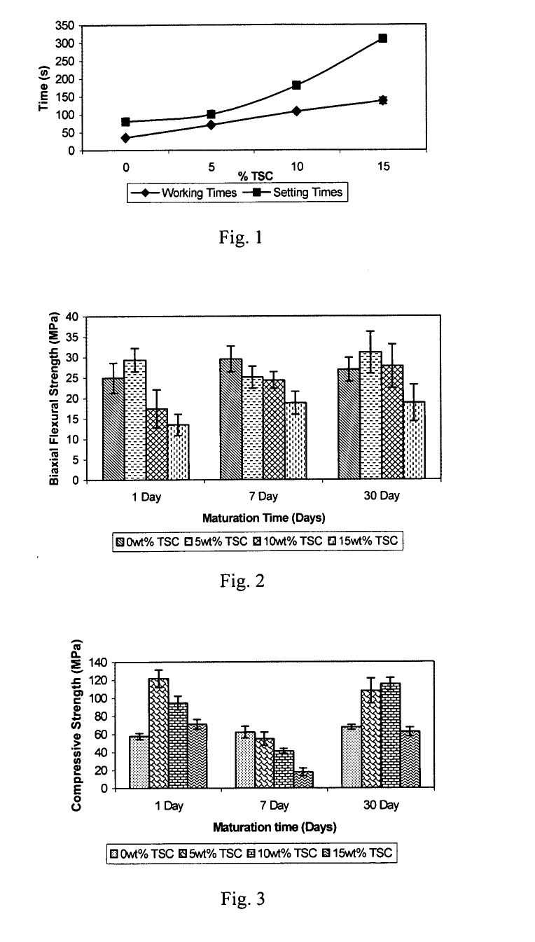

Controlled addition of TSC significantly improves the rheology of the bone cements. Increases in working and setting times with increasing TSC content are illustrated in FIG. 1. It can be seen that for the three formulations above of Zn-GPC the working time can be adjusted from less than 50 seconds to almost 120 seconds; concomitantly working times improve from 58 seconds to duration in excess of 300 s. Such working times now indicate clearly that the Zn-GPCs modified with TSC are suitable for orthopedic applications.

The TSC is a biocompatible agent which chelates ions released from the glass network Citrate forms complexes with GPC matrix-forming ions, and such complexes inhibit the formation of stable metal polyacrylate anion complexes, thus retarding the setting reaction. Such effects account for the observed increases in working time and setting time associated with increasing TSC content.

### Equivalent Strength Characteristics

Controlled additions of TSC to Zn-GPCs do not compromise strength. Samples of GPCs of the invention were subjected to biaxial flexural strength (BFS) testing and compressive strength (CS) testing. The full cement composition is BT100 50 wt % GPC with 0, 5, 10 and 15 wt % TSC addition. The results of these tests (FIGS. 2 and 3) were subjected to one-way ANOVA analysis and post-hoc Bonferroni tests to determine the effect of TSC addition and the results are presented in Tables 4 and 5 (the mean difference is significant at values less than 0.05, i.e. P<0.05). From the results it can be seen that the addition of 5 and 10 wt % TSC does not significantly alter the biaxial flexural strength of the GPCs formulated. However, at 15 wt % TSC addition, a significant decrease in BFS is observed. Nevertheless, the results illustrate that additions of TSC can be tailored to increase the working times of the materials contained herein without adversely affecting BFS.

TABLE 4
Multiple comparison (Bonferroni) of mean compressive strengths.
Mean difference is significant at the 0.05 level.

| Means compared | 1 Day | 7 Day | 30 Day |
|---|---|---|---|
| 0 wt %/5 wt % | P < 0.000 | P < 1.000 | P < 0.000 |
| 0 wt %/10 wt % | P < 0.000 | P < 0.000 | P < 0.000 |
| 0 wt %/15 wt % | P < 0.049 | P < 0.000 | P < 0.000 |

After one day, compressive strengths were significantly greater than that of the control material. Indeed, 5 wt % addition of TSC resulted in an increase greater than 100% (from 58 MPa to 122 MPa).

TABLE 5
Multiple comparison (Bonferroni) of mean biaxial flexural strengths.
Mean difference is significant at the 0.05 level.

| Means compared | 1 Day | 7 Day | 30 Day |
|---|---|---|---|
| 0 wt %/5 wt % | P < 0.289 | P < 0.172 | P < 1.000 |
| 0 wt %/10 wt % | P < 0.091 | P < 0.056 | P < 1.000 |
| 0 wt %/15 wt % | P < 0.002 | P < 0.00 | P < 0.073 |

However, mean compressive strength (CS) of each group decreases with greater addition of TSC. However, at one day, all strengths are still equivalent to, or greater than, the unmodified Zn-GPC. After seven days the control materials and the GPC modified with 5 wt % TSC addition have comparable strengths.

In summary, it is possible to extend the working times and setting times of Zn-GPC without causing a significant change in mechanical properties. This advancement is possible through the controlled addition of TSC into the Zn-GPC formulation.

### Additional Benefits

### Antibacterial Activity

Table 6 below states inhibition zones from antibacterial analysis of one cement (BT101/E9 50 wt %), with respect to TSC content, when analysed on agar diffusion assays swabbed with Ecoli bacteria. A 350-μl volume of each bacterial suspension was streaked using clinical swabs on MH agar plates containing agar of 4 mm height, following which 2-3 discs of each material were placed on the agar. Baseline samples were mixed and allowed to set for one hour (37C.) then placed in contact with the bacteria. The plates were inverted and incubated under aerobic conditions (36 h, 37° C.). Callipers were used to measure zones of inhibition at three different diameters for each disc and zone sizes were calculated as follows:

Size of inhibition zone(mm)=(haloØ−discØ)/2.

All cements were analysed in triplicate and average mean zone sizes were calculated. For the 1, 7 and 14 day samples the cements were incubated in distilled water for these time frames prior to contact with the agar assay. The cements containing no TSC also exhibit an antibacterial nature due to the release of strontium and zinc ions from the cement mantle

TABLE 6
mean inhibition zones of TSC containing versions of
BT101/E9 50 wt % cements

| TSC | | | | |
|---|---|---|---|---|
| Wt % | Baseline | 1 Day | 7 Day | 14 Day |
| 0 | 4 | 2 | 2 | 1.9 |
| 5 | 7.1 | 2.3 | 3 | 2.3 |
| 10 | 10.2 | 3.4 | 3.5 | 3 |
| 15 | 12.9 | 4.3 | 4.2 | 3.8 |

TSC is of particular interest as an additive because citrate ions are present in bone mineral. TSC can reduce the viscosity of calcium phosphate cement (CPC) pastes at lower concentrations. We have demonstrated that the controlled addition of TSC to the Zn-GPC formulations can significantly improve the rheological properties. Such improvements indicate that these materials, correctly modified with TSC, can be considered for a multitude of orthopaedic applications.

While TSC is the main example employed, it is expected that beneficial results would be achieved using other water soluble citrate (C₆H₅O₂) salts such as calcium citrate. The reason these would also be of benefit is that such agents are biocompatible and chelate ions released from the glass network during setting.

Also, it is possible to mix a version of this cement by partially or totally replacing the water in the reagents with blood, saline, saliva or other body fluids or synthetic alternatives (such as simulated body fluid). Blood, for example, is composed of 85 wt % water. Calcium phosphate bone cements can be formulated with the patient's blood replacing the aqueous content in part or whole.

The invention is not limited to the embodiments described but may be varied in construction and detail. For example, in other embodiments, the TSC (or other rheology-improving agent) could be incorporated in the glass, either as a network former or modifier. Also, although TSC is described as being used in a powder form, an aqueous version of TSC could be used where it is suspended in the water and kept separate from the powder components (i.e. glass and acid) until ready for mixing.

1. A bone or dental cement comprising a glass and an acid, wherein the cement comprises a biocompatible agent capable of chelating ions released from the glass network during a setting process.

2. A cement as claimed in claim 1, wherein the agent comprises a surfactant.

3. A cement as claimed in claim 1, wherein the agent comprises a water soluble citrate salt.

4. A cement as claimed in claim 1, wherein the agent comprises trisodium citrate, Na₃C₆H₅O₇.

5. A cement as claimed in claim 1, wherein the agent comprises calcium citrate.

6. A cement as claimed in claim 1, wherein the agent is present in a proportion of up to 30 wt %.

7. A cement as claimed in claim 6, wherein the agent is present in a proportion of 5 wt % to 10 wt %.

8. A cement as claimed in claim 1, wherein the glass comprises zinc as either a network former or a network modifier.

9. A cement as claimed in claim 8, wherein the glass comprises ZnO.

10. A cement as claimed in claim 1, wherein the glass comprises SrO.

11. A cement as claimed in claim 1, wherein the glass comprises CaO.

12. A cement as claimed in claim 1, wherein the glass comprises SiO₂.

13. A cement as claimed in claim 1, wherein the acid comprises a water soluble polyalkenoic acid.

14. A cement as claimed in claim 13, wherein the acid is poly-acrylic acid.

15. A cement as claimed in claim 13, wherein the acid is present at a concentration in the range of 20% m/w to 60% m/w.

16. A cement as claimed in claim 1, wherein the agent is separate from the glass.

17. A cement as claimed in claim 1, wherein the agent is incorporated in the glass.

18. A method of producing a bone cement, the cement having a target setting time, the method comprising the steps of producing a cement of claim 1 in which the agent concentration is chosen according to the target setting time.

19. A method of producing a bone cement, the cement having a target working time, the method comprising the steps of producing a cement of claim 1 in which the agent concentration is chosen according to the target working time.

20. A method as claimed in claim 18, in which the agent is added as a powder to the glass and the acid before mixing with water.

21. A method as claimed in claim 19, in which the agent is added as a powder to the glass and the acid before mixing with water.

22. A bone or dental cement comprising a glass and an acid, wherein the cement comprises a biocompatible agent capable of chelating ions released from the glass network during a setting process, wherein said agent comprises trisodium citrate, Na₃C₆H₅O₇.

23. A cement as claimed in claim 22, wherein the agent is present in a proportion of 5 wt % to 10 wt %.

24. A cement as claimed in claim 22, wherein the glass comprises ZnO, SrO, CaO, and SiO₂.

25. A cement as claimed in claim 22, wherein the acid comprises a water soluble polyalkenoic acid.
